(19) United States
(12) Patent Application Publication
(10) Pub. No.: US 2017/0290968 A1
(43) Pub. Date: Oct. 12, 2017

(54) MEDICAL DEVICE COMPRISING MONITOR SYSTEM

(71) Applicant: B. BRAUN AVITUM AG, MELSUNGEN (DE)

(72) Inventors: OLIVER SCHÄFER, NUENSTEIN (DE); CHRISTIAN SCHLEICHER, DIPPERZ (DE)

(21) Appl. No.: 15/480,789

(22) Filed: Apr. 6, 2017

(30) Foreign Application Priority Data
Apr. 12, 2016 (DE) 10 2016 106 655.9

(51) Int. Cl. A61M 1/14

(52) U.S. Cl. CPC A61M 1/14; A61M 2205/505; G06F 3/0488

(57) ABSTRACT

A medical device including a body and a monitor system constituting a display and/or operating unit which includes a curved display and/or user interface. The medical device is preferably designed as an apparatus for extracorporeal blood treatment and, more preferred, as a dialysis machine including a housing constituting the body for accommodating operational components for the extracorporeal blood treatment. The display and/or user interface of the monitor system is designed to be concavely or convexly curved about at least one space axis, preferably a vertically or horizontally extending axis.

### CROSS REFERENCE TO RELATED APPLICATIONS

This application claims priority to German application DE 10 2016 106 655.9 filed Apr. 12, 2016, the contents of such application being incorporated by reference herein.

### FIELD OF THE INVENTION

The invention relates to a medical device comprising a monitor system and especially relates to an apparatus for extracorporeal blood treatment such as a dialysis machine including a monitor system, for example in the form of a touch screen.

### BACKGROUND OF THE INVENTION

In particular, in the case of stationary monitor subassemblies which cannot be rotated or tilted these solutions are a drawback to the effect that a dazzle effect may considerably impair the visibility and/or readability of the shown content of the monitor.

### SUMMARY OF THE INVENTION

Therefore, the object underlying the invention is to provide a medical device comprising a monitor system which reduces reflections and/or enhances non-glaring in a stationary monitor system and provides improved visibility and/or readability of contents shown on the monitor system.

Especially for an apparatus for extracorporeal blood treatment, in particular a dialysis machine, a monitor system of this type is to be provided.

This object is achieved, according to aspects of the invention, by a medical device comprising the features of the independent claims. Advantageous further developments of the invention are the subject matter of the dependent claims.

The invention relates to the general idea to arrange, on a device for medical engineering and especially on an apparatus for extracorporeal blood treatment such as a dialysis machine and the like, a monitor system which is bent, arched or curved, for example concavely with respect to a viewer/operator/user in the form of e.g. a screen having an appropriate shape of a display and/or user interface including a graphical user interface, where necessary. A touch-sensitive film put onto the monitor surface may permit, in combination with the display panel located there beneath, for example a TFT system, both a graphical display of parameters relevant to the therapy and the adjustment of various parameters. The concavely designed monitor system (in the form of a concave display) definitely reduces dazzle effects. A concave monitor surface including an integrated touch film is further advantageous to the effect that it is not only dazzle effects that are reduced thus enabling a more convenient representation of information due to the reduced dazzle effects, but also the risk of possible surface damage of the display is reduced.

In one embodiment, the concavity of the surface can be adapted to the typical distance of the user from the surface of the monitor. In this case, by virtue of the curved shape of the monitor surface the light is not reflected to the eye of the user but is scattered in all directions. At the same time, in the case of illumination from the rear view of the user the body of the latter casts a shadow onto the concave monitor which has a contrast-enhancing effect. The contrast may be even further enhanced by the use of OLED technology in combination with a curved display.

Particular advantages are resulting from an array of devices and, respectively, machines having a stationary monitor, as in this case rotation of the monitor with respect to a disturbing light source is restricted, as for rotation of the monitor the entire machine has to be rotated. In so far, with a concave monitor a rotary function of the monitor may be dispensed with, as unfavorable light incidence onto the monitor will be scattered and therefore will not disturb the user.

In detail, the afore-mentioned advantages are realized and the object is achieved by a medical device having a body and a monitor system in the form of a display and/or operating means which includes a curved display and user interface.

Preferably the display and/or user interface is evenly or continuously curved.

Of preference, the medical device is designed as an apparatus for extracorporeal blood treatment comprising a housing constituting the body for accommodating functional components for the extracorporeal blood treatment. Further preferred, the medical device is a dialysis machine.

The display and/or user interface is preferably configured to be curved concavely or convexly about at least one space axis. Accordingly, the space axis may be at least one horizontally or vertically extending space axis.

Further preferred, the display and/or user interface of the monitor system may include a plurality of concavely or convexly curved segments.

The display and/or operating unit may preferably be part of a monitor system being fixed to a surface of the housing and projecting from the latter.

Further preferred, the display and/or operating unit and, respectively, the display and/or user interface may be integrated in the housing.

Preferably a plurality of monitor systems each including at least one display and/or user interface may be provided and at least one monitor system may be a monitor system which is externally linked to the housing or which is integrated in the housing.

Preferably the display and/or operating unit is a display and/or operating unit which functions on the basis of the OLED or AMOLED principle.

Preferably the concavity or the convexity of the curvature of the display and/or operating unit is adapted to a predetermined distance of a user from the device. In particular, the distance is within an operating range and, respectively, within an average armlength of a user, preferably within the range of from 0.2 m to 1 m, especially preferred within a range of from 0.3 m to 0.7 m.

Further preferred, the degree and/or the course of the curvature of the display and/or user interface is provided and adapted for reducing reflections of light beams at a predetermined distance of the user from the display and/or user interface toward the eye of the user.

Preferably the degree and/or the course of the curvature of the display and/or user interface is provided and adapted for scattering light beams, especially by using a structured layer which is arranged ahead of the display and/or user interface and assists the scattering effect.

### BRIEF DESCRIPTION OF THE DRAWINGS

The invention is best understood from the following detailed description when read in connection with the accompanying drawings. Included in the drawings are the following figures:

FIG. 1A shows in a simplified perspective view an apparatus housing of a dialysis machine constituting a medical device for extracorporeal blood treatment comprising a display and/or operating unit arranged externally on a body;

FIG. 1B shows the display and/or operating unit in a modified arrangement and, respectively, orientation according to a first embodiment; and

FIG. 2 shows in a simplified perspective view an apparatus housing of a dialysis machine constituting a medical device for extracorporeal blood treatment comprising a display and/or operating unit arranged internally within a body according to a second embodiment.

### DETAILED DESCRIPTION OF THE PREFERRED EMBODIMENTS

In the following description of figures, like or equally acting elements and/or components are designated equally and/or with like reference numerals and adequately are not described redundantly. In cases in which a subsequent embodiment as to function corresponds to at least a preceding one, i.e. corresponding functions, arrangements and/or process or operating flows are equally comprised, adequately only differences will be discussed.

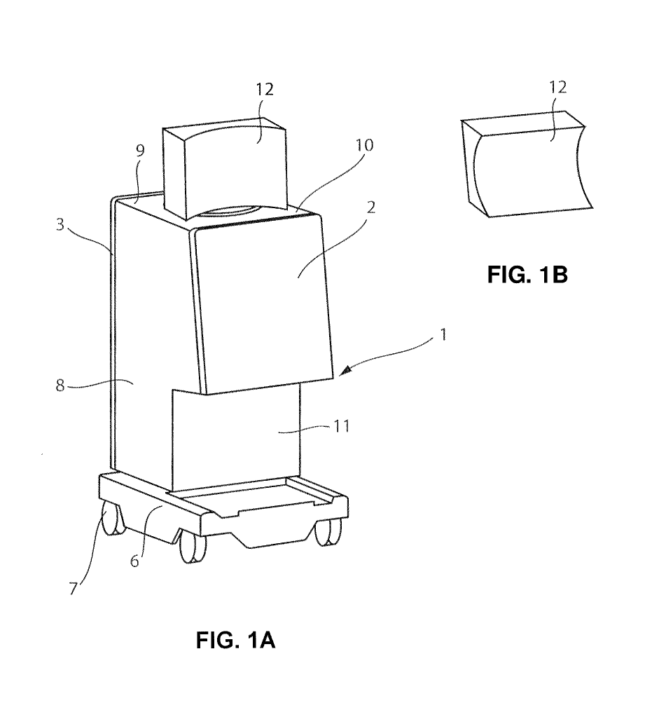

FIG. 1A illustrates in a simplified perspective representation an apparatus housing of a dialysis machine.

According to FIG. 1A, the dialysis machine has an apparatus or machine housing 1 (hereinafter referred to as body or housing) in which a number of components for carrying out blood treatment are or can be arranged in a manner known per se to be functioning by interaction, such as control electronics, at least parts of the hydraulic system, pumps, heating units and, where necessary, tanks or bags for selected solutions, all of which are sufficiently known from the state of the art and therefore will not be illustrated or described in greater detail.

The housing 1 may substantially include, for example, at least two side plates 8, a rear wall 3 at least partially configured as a sliding door/flap, a hood 9 connecting the two side plates 9 preferably spaced in parallel on the upper side, a bottom plate (for example as part of an apparatus base) 6 connecting the two side plates 8 on the bottom side and a front wall at least partially configured as a sliding door/flap 2.

On the bottom plate 6 apparatus rolls 7 which, together with the bottom plate 6, may form an internal (integrated) carriage of a mobile dialysis machine may be mountable. Moreover, the hood 9 and the apparatus base and, respectively, the bottom plate 6 can be fixed to the upper and lower edges of the two side plates 8, for example welded for stiffening over at least parts of the edge length. In this way, a torsion-resistant housing 1 the inside of which is accessible via parts to be opened can be provided by the two side plates 8, the hood 9, the bottom plate 6 and a front plate 11.

In this embodiment, a connecting base 10 on which a monitor system in the form of a monitor is mounted is centrally formed integrally with the hood 9. The monitor system may be designed to have a display and/or user interface 12 for the mere display of information or may alternatively (not shown) be a display and/or user interface which includes at least one information display portion and at least one operating portion (graphical user interface) comprising a number of operating elements for operating the machine. Preferably in this case at least part of the monitor system is formed to be touch-sensitive, for example with a resistively or capacitively touch-sensitive film or layer located above a thin-film transistor arrangement. Touch-sensitive display and/or operating units are known per se and therefore will not be described in greater detail.

In this embodiment, furthermore the total monitor system, but at least part of its display and/or user interface 12 is formed to be bent or curved concavely, i.e. away from a user standing in front of the apparatus, as shown in FIG. 1A, about a space axis, preferably about a height direction and, respectively, about a vertically extending axis.

FIG. 1B illustrates the display and/or operating unit in a modified arrangement and, respectively, orientation according to the first embodiment. In accordance with FIG. 1B, the monitor system may be designed concavely in an arrangement and, respectively, orientation modified with respect to FIG. 1A about a different space axis, for example in the lateral direction and, respectively, about a horizontally extending axis.

In a different modification, which is not shown, the monitor system may as well include plural sections each being separately curved, for example plural concave sections and/or convex sections at a predetermined arrangement or distribution, for example in wave shape, over the display and/or user interface 12 of the monitor system. In another modification, which is not shown, the monitor system moreover may be concavely curved both in the height direction and in the lateral direction starting from predetermined points, for example the corner points of a rectangular display and/or user interface 12, in the type of a trough drawn toward the user at said points and lowered in the residual area.

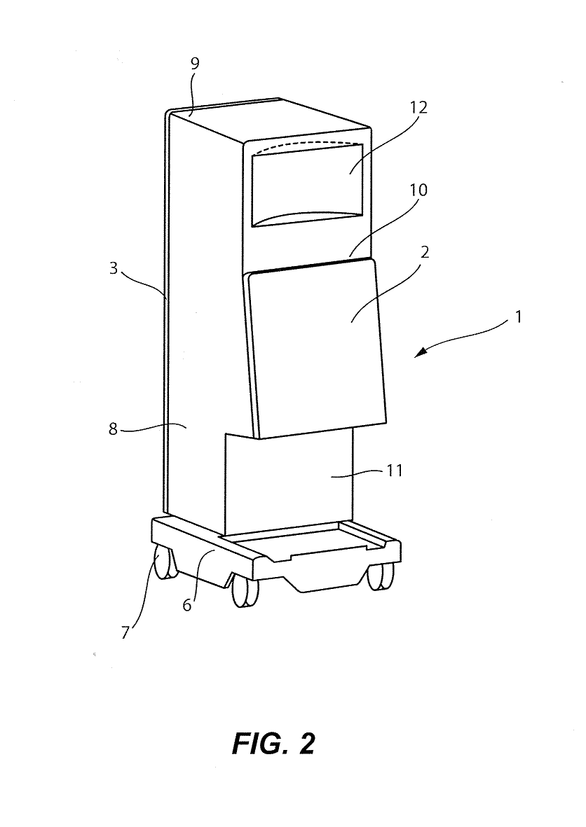

FIG. 2 illustrates in a simplified perspective representation an apparatus housing of a dialysis machine in the form of a medical device for extracorporeal blood treatment comprising a display and/or operating unit arranged internally within a body according to a second embodiment.

In the second embodiment according to FIG. 2, the monitor comprising the display and/or operating unit 12 is not mounted to the upper side of the body of the housing 1 but is integrated in the body of the housing 1 and is fixed to the backside of the front plate 11 at a predetermined position with a frame means or retaining means (support). In the second embodiment, thus the same configurations, embodiments and modifications are possible as in the first embodiment.

Moreover, in the first and second embodiments preferably the degree and the shape and, respectively, the course of the concavity of the display and/or user interface 12 may be adapted to a predetermined distance of the user from e.g. a surface of the monitor receiving or including the display and/or user interface 12 in a way enhancing the contrast such that due to the curved shape of the surface light reflects to a smaller extent to the eye of the user but is rather scattered in a diversifying manner. For enhancing the scattering effect, an appropriately structured, preferably transparent or appropriately opaque cover layer, filter film or the like may be arranged ahead of the surface of the monitor system.

The arrangement of a light source behind the user may further enhance the contrast by light which is incident past the user and a shadow cast by the user's body. An additional enhancement of contrast may further be achieved by preferably using the OLED or AMOLED technology known per se in combination with the curved surface of the monitor or the monitor system, respectively.

According to the first and second embodiments, advantages are especially resulting from an arrangement of the curved monitor system on apparatuses and, respectively, machines having a fixed or integrated monitor, as in machines of this type rotation of the monitor relative to a disturbing light source is restricted, because in this case the entire machine would have to be rotated. In so far, a rotary function of the monitor can be dispensed with due to the arrangement of a concavely formed monitor, as light incidence on the monitor which is reflecting in an unfavorable or disturbing manner is scattered in a way reducing reflections and/or dazzle effects and not disturbing the user.

As described in the foregoing, according to the first embodiment a predetermined and/or adapted monitor system subassembly may be provided which can be attached to an (already existing and thus retrofittable) housing 1. According to the second embodiment, a stationary monitor can be directly integrated in a housing 1. Moreover, at least parts of the first and second embodiments can be combined. For example, an (additional and/or external) secondary or sub-monitor is imaginable which may be integrated at a different position, for example, or may be disposed on the housing 1 with a possibly pivoting arm device.

It is noted that comparable effects and advantages may also be achieved by a surface of the monitor system being curved convexly, i.e. towards the user.

In the foregoing, thus a medical device comprising a body and a display and/or operating unit which includes a curved display and/or user interface 12 has been described. The medical device preferably is an apparatus for extracorporeal blood treatment and, more preferred, a dialysis machine comprising a housing 1 constituting the body for receiving operational components for the extracorporeal blood treatment. The display and/or user interface 12 is designed to be curved concavely or convexly about at least one space axis, preferably a vertically or horizontally extending space axis.

It is understood that the invention is not limited to the afore-described embodiments but that within the scope of protection defined by the following claims combinations of at least parts of these embodiments, modifications and equivalents may nonetheless be obvious to those skilled in the art.

1.-9. (canceled)

10. A medical device comprising:
a body; and
a monitor system constituting at least one of a display or operating unit which includes at least one of an evenly curved display or user interface.

11. The medical device according to claim 10 wherein the body is a housing for accommodating operational components for extracorporeal blood treatment.

12. The medical device according to claim 10, wherein the at least one display or user interface is at least one of concavely or convexly curved about at least one horizontally or vertically extending axis.

13. The medical device according to claim 10, wherein the at least one display or user interface includes a plurality of separately concavely or convexly curved segments.

14. The medical device according to claim 11, wherein the at least one display or user interface is part of a monitor system fixed to a surface of the housing.

15. The medical device according to claim 11, wherein the at least one display or user interface is integrated in the housing.

16. The medical device according to claim 10, wherein the monitoring system comprises a plurality of monitor systems, each of the plurality of monitoring systems includes at least one display or user interface, and at least one of the plurality of monitor systems is externally linked to the housing or is integrated in the housing.

17. The medical device according to claim 10, wherein the at least one display or operating unit functions on the basis of the OLED or AMOLED principle.

18. The medical device according to claim 12, in which the concavity or the convexity of the curvature of the at least one display or user interface is adapted to a predetermined distance of a user from the at least one of display or user interface.

19. An apparatus for extracorporeal blood treatment, the apparatus comprising:
the medical device of claim 10.

20. The apparatus of claim 19, wherein the extracorporeal blood treatment apparatus is a dialysis machine.
